(19) United States
(12) Patent Application Publication
(10) Pub. No.: US 2006/0129097 A1
(43) Pub. Date: Jun. 15, 2006

(54) Safe syringe

(76) Inventors: Jih-Hsiung Yang, Taichung Hsien (TW); Hsiu-Chih Lin, Taichung Hsien (TW)

Correspondence Address: DELLETT & WALTERS, P. O. BOX 82788, PORTLAND, OR, 97282-0788, US

(21) Appl. No.: 10/999,619

(22) Filed: Nov. 29, 2004

(51) Int. Cl. A61M 5/00

(52) U.S. Cl. 604/110; 604/195

(57) ABSTRACT

A safe syringe has a barrel, a needle assembly, a sheath and a plunger. The barrel has an open front end, an open back end and multiple longitudinal slots defined through the barrel and communicate with the open front end. The needle assembly is mounted retractably in the open front end of the barrel and has a tapered hub, a needle and a plug. The tapered hub has a front, an open back, an axial recess and multiple longitudinal grooves. The needle is mounted on the tapered hub. The plug is mounted slidably into the axial recess. The sheath is mounted around the open front end of the barrel. The plunger is mounted slidably through the open back end of the barrel. The needle assembly is pulled into the barrel by a vacuum force generated by the plunger so medical personnel can safely deal with the used safe syringe.

### BACKGROUND OF THE INVENTION

1. Field of the Invention

The present invention relates to a syringe, and more particularly to a safe syringe that prevents medical personnel from being injured by a syringe needle when medical personnel handle and dispose of used syringes.

2. Description of Related Art

Syringes are generally used to inject liquid medication into a human body. Conventional syringes usually have a barrel, a plunger and a needle cap. The barrel has a front end, an open back end, a medication chamber and a needle. The medication chamber is defined in the barrel and communicates with the open back end. The needle is hollow, is mounted through the front end of the barrel and has a longitudinal passage that communicates with the medication chamber. The plunger is mounted slidably through the open back end and into the medication chamber and has a front end, a back end, a rod and a sealed piston. The sealed piston is mounted on the front end of the plunger and slidably inside the medication chamber. The needle cap is mounted detachably on the front end of the barrel and covers the needle. After the syringe is used, medical personnel such as doctors or nurses put the needle cap back on the barrel so the needle of the used syringe is covered and will not injure another person accidentally. However, the medical personnel may be stabbed inadvertently by the contaminated needle when the needle cap is reinstalled on the front of the barrel and contract a disease.

To overcome the shortcomings, the present invention provides a safe syringe to mitigate or obviate the aforementioned problems.

### SUMMARY OF THE INVENTION

The main objective of the invention is to provide a safe syringe that prevents medical personnel from being hurt by contaminated needles when medical personnel handle contaminated syringes.

A safe syringe in accordance with the present invention comprises a barrel, a needle assembly, a sheath and a plunger.

The barrel has an open front end, an open back end and multiple longitudinal slots. The longitudinal slots are formed through the barrel and communicate with the open front end.

The needle assembly is mounted retractably in the open front end of the barrel and has a tapered hub, a needle and a plug. The tapered hub has a front, an open back, an axial recess and multiple longitudinal grooves. The needle is mounted on the tapered hub. The plug is mounted slidably in the axial recess.

The sheath is mounted around the open front end of the barrel.

The plunger is mounted slidably through the open back end of the barrel.

Other objectives, advantages and novel features of the invention will become more apparent from the following detailed description when taken in conjunction with the accompanying drawings.

### BRIEF DESCRIPTION OF THE DRAWINGS

FIG. 1 is an exploded perspective view of a safe syringe in accordance with the present invention;

FIG. 2 is a cross sectional side view of the safe syringe in FIG. 1;

FIG. 3 is a cross sectional front view of the safe syringe in FIG. 1;

FIG. 4 is an operational cross sectional side view of the safe syringe in FIG. 2 with the plug pressed completely into the axial recess in the tapered hub;

FIG. 5 is an operational cross sectional side view of the safe syringe in FIG. 2 with the sheath rotating to release the end tabs on the barrel; and

FIG. 6 is an operational cross sectional side view of the safe syringe in FIG. 2 with the plunger broken at the optional annular snapping groove.

### DETAILED DESCRIPTION OF PREFERRED EMBODIMENT

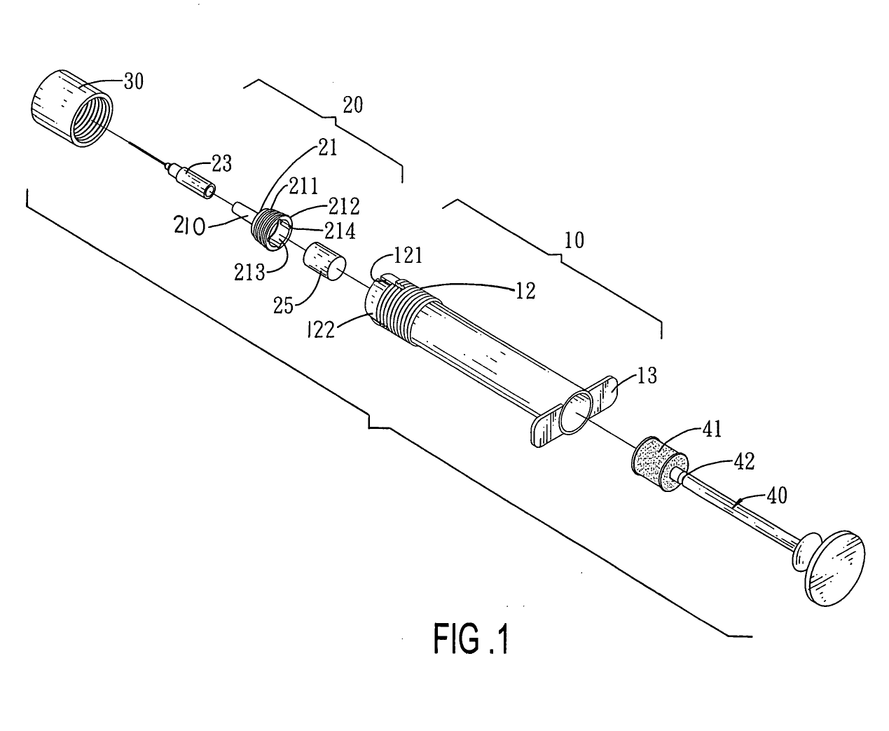

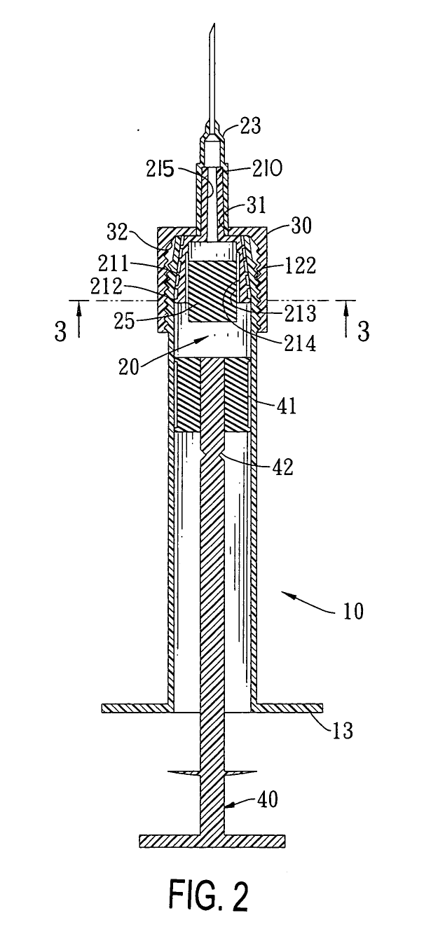

With reference to FIGS. 1 and 2, a safe syringe in accordance with the present invention comprises a barrel (10), a needle assembly (20), a sheath (30) and a plunger (40).

The barrel (10) is made of resilient material such as plastic and has an open front end, a open back end, a medication chamber, an outer surface, an inner surface, an outer thread (12), multiple longitudinal slots (121), multiple end tabs (122) and two opposite finger tabs (13).

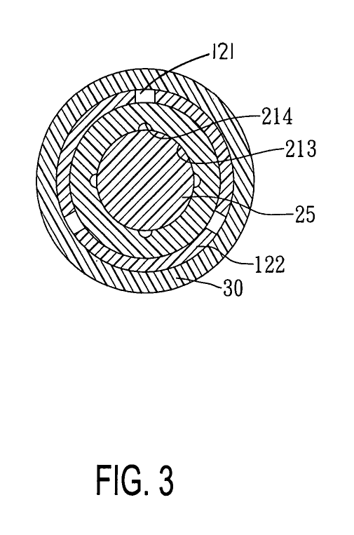

The medication chamber is defined inside the barrel (10) and communicates with the open front end and the open back end. The outer thread (12) is formed on the outer surface close to the open front end of the barrel (10). The longitudinal slots (121) are defined through the open front end and partially through the outer thread (12) at intervals. Each longitudinal slot (121) has a length. The end tabs (122) are defined respectively between adjacent longitudinal slots (121). With further reference to FIG. 3, a preferred embodiment of the barrel (10) has three longitudinal slots (121) and three end tabs (122). The finger tabs (13) extend out radially from the open back end of the barrel (10).

The needle assembly (20) is mounted retractably through the open front end of the barrel (10) and has a tapered hub (21), a needle (23) and a plug (25).

The tapered hub (21) is mounted slidably in the open front end of the barrel (10) and has a tapered front, an open back, an outer surface, an inner surface, a length, a mounting tube (210), a central hole (215), multiple optional annular nonskid ribs (211), an annular flange seal (212), an axial recess (213) and multiple longitudinal grooves (214).

The open back of the tapered hub (21) communicates with the medication chamber in the barrel (10). The length of the tapered hub (21) is longer than the length of the longitudinal slots (121) in the barrel (10). The mounting tube (210) is formed on the tapered front of the tapered hub (21). The central hole (215) is defined through the mounting tube (210) and the tapered front of the tapered hub (21). The annular nonskid ribs (211) are formed on the outer surface of the tapered hub (21) at intervals. The annular flange seal (212) extends out radially from the open back of the tapered hub (21), abuts the inner surface of the barrel (10) and forms a seal between the barrel (10) and the tapered hub (21). The axial recess (213) is defined inside the tapered hub (21), communicates with the central hole (215) and the open back and has a depth. The multiple longitudinal grooves (214) are defined longitudinally in the inner surface of the tapered hub (21) and communicate with the axial recess (213) and the open back. Each longitudinal groove (214) has a length shorter than the depth of the axial recess (213). A preferred embodiment of the tapered hub (21) has four longitudinal grooves (214).

The needle (23) is mounted on the mounting tube (210) of the tapered hub (21) and has a needle passage. The needle passage is defined axially through the needle and communicates with the central hole (215) in the mounting tube (210).

The plug (25) is cylindrical, corresponds to and is mounted slidably in the axial recess (213) in the tapered hub (21), extends through the open back of the tapered hub (21) and has a length equal to the depth of the axial recess (213).

The sheath (30) is mounted detachably around the open front end of the barrel (10) and has a disk, a sidewall, a through hole (31), an inner thread and an inclined annular surface (32).

The disk of the sheath (30) abuts the end tabs (122) of the barrel (10) and has an outer edge. The sidewall is formed perpendicularly around the outer edge of the disk of the sheath (30) and has an inner surface. The through hole (31) is defined through the disk and allows the mounting tube (210) of the tapered hub (21) to extend through the through hole (31). The inner thread is formed on the inner surface of the sidewall, corresponds to and screws onto the outer thread (12) on the barrel (10). The inclined annular surface (32) is defined inside the sheath (30) between the disk and the sidewall. The inclined annular surface (32) presses the end tabs (122) of the barrel (10) inward, so the end tabs (122) abut the annular nonskid ribs (211) on the tapered hub (21).

The plunger (40) is mounted slidably through the open back end of the barrel (10) and has a front end, a back end, an outer surface, an optional annular snapping groove (42) and a sealed piston (41). The annular snapping groove (42) is defined in the outer surface close to the front end of the plunger (40). The sealed piston (41) is made of resilient material such as rubber, is mounted securely around the front end of the plunger (40) and is mounted slidably in the medication chamber in the barrel (10).

The new syringe in accordance with the present invention is in a condition that the plug (25) is located incompletely in the axial recess (213) of the tapered hub (21), as shown in FIG. 2 so the central hole (215) in the mounting tube (210) communicates with the longitudinal grooves (214) and the medication chamber in the barrel (10).

When drawing blood or liquid medication, the plunger (40) is pulled back so the blood or liquid medication passes through the needle passage in the needle (23), the central hole (215) in the mounting tube (210), the axial recess (213) and the longitudinal grooves (214) in the tapered hub (21) and finally into the medication chamber in the barrel (10). Then, the needle (23) is oriented upward and the plunger (40) is pushed slightly into the barrel (10) to discharge the remaining air out from the barrel (10).

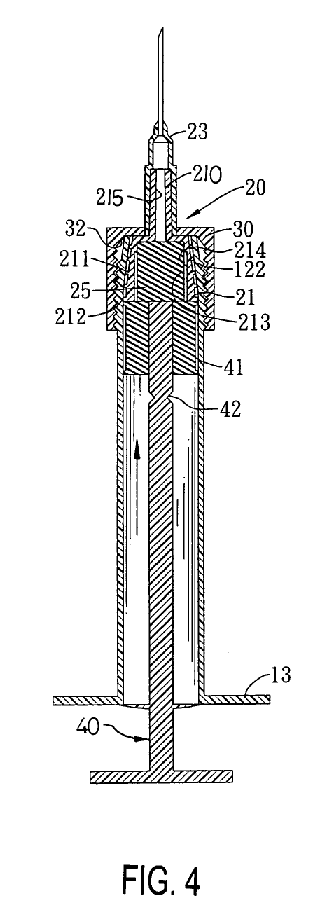

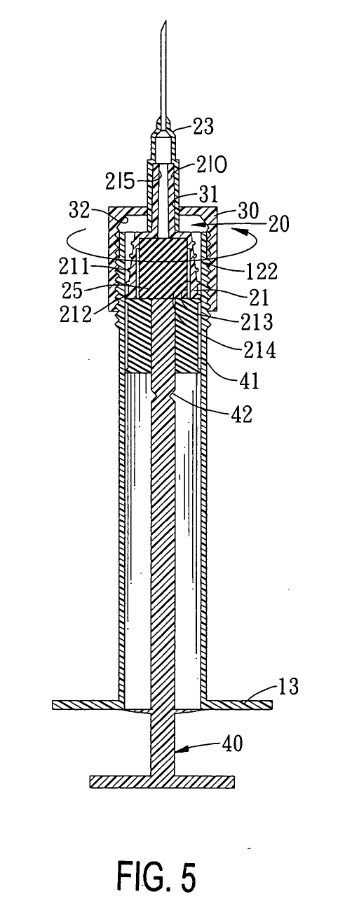

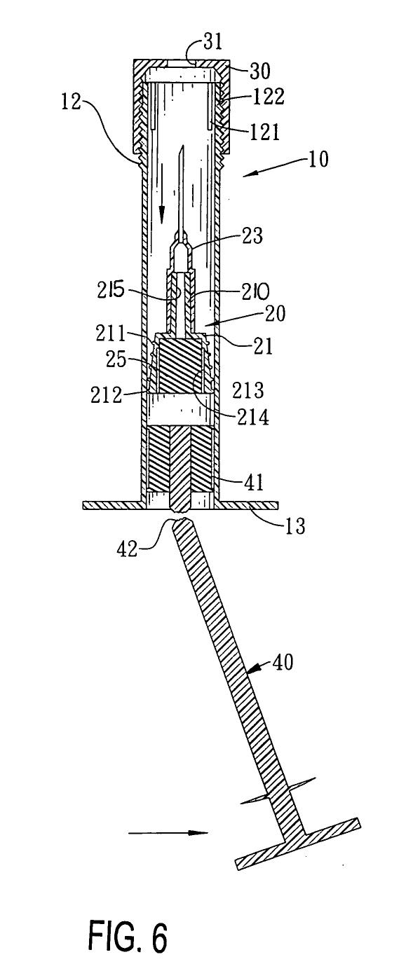

With reference to FIGS. 4 to 6, the safe syringe is made safe and discarded after use. When the safe syringe inject medication into a patient, the front end of the plunger (40) presses the plug (25) completely into the axial recess (213) of the tapered hub (21) as the last of the medication is injected into the patient. The sealed piston (41) abuts and seals the open back end of the tapered hub (21) and closes the longitudinal grooves (214) so air inside the longitudinal grooves (214) is sealed. The central hole (215) in the mounting tube (210) is separated from the longitudinal grooves (214). The sheath (30) is rotated to separate the inclined annular surface (32) from the end tabs (122) of the barrel (10) so the end tabs (122) separate from the annular nonskid ribs (211) on the tapered hub (21). The plunger (40) is pulled away from the barrel (10) and simultaneously draws the needle assembly (20) into the barrel (10) with a vacuum inside the barrel (20), wherein the vacuum derives from the sealed inexpansible blood or liquid medication between the tapered hub (21) and the sealed piston (41). When the annular snapping groove (42) reaches the open rear end of the barrel (20), the plunger (40) is snapped off at the annular snapping groove (42) to prevent the needle assembly (20) from extending out of the open front end of the barrel (10).

With the needle assembly (20) retracted into the barrel (10), medical personnel can safely deal with the used safe syringe without fear of injury from the contaminated needle.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A safe syringe comprising:
a barrel made of resilient material and having
an open front end;
an open back end;
a medication chamber defined inside the barrel and communicating with the open front end and the open back end;
an outer surface;
an inner surface;
an outer thread formed on the outer surface close to the open front end of the barrel;
multiple longitudinal slots defined through the open front end and partially through the outer thread at intervals, and each longitudinal slot having a length;
multiple end tabs defined respectively between adjacent longitudinal slots; and
two opposite finger tabs extending out radially from the open back end of the barrel;
a needle assembly mounted retractably through the open front end of the barrel and having
a tapered hub mounted slidably in the open front end of the barrel and having
a tapered front;
an open back communicating with the medication chamber in the barrel;
an outer surface;
an inner surface;
a length longer than the length of the longitudinal slots in the barrel;
a mounting tube formed on the tapered front of the tapered hub;
a central hole defined through the mounting tube and the tapered front of the tapered hub;
an annular flange seal extending radially out from the open back of the tapered hub, abutting the inner surface of the barrel and forming a seal between the barrel and the tapered hub;
an axial recess defined inside the tapered hub, communicating with the central hole and the open back and having a depth; and
multiple longitudinal grooves defined longitudinally in the inner surface of the tapered hub and communicating with the axial recess and the open back, and each longitudinal groove having a length shorter than the depth of the axial recess;
a needle mounted on the mounting tube of the tapered hub and having a needle passage defined axially through the needle and communicating with the central hole in the mounting tube; and
a plug corresponding to and mounted slidably into the axial recess in the tapered hub, extending through the open back of the tapered hub and having a length equal to the depth of the axial recess;
a sheath mounted detachably around the open front end of the barrel and having
a disk abutting the end tabs of the barrel and having an outer edge;
a sidewall formed perpendicularly around the outer edge of the disk of the sheath and having an inner surface;
a through hole defined through the disk and allowing the mounting tube of the tapered hub to extend through the through hole;
an inner thread formed on the inner surface of the sidewall, corresponding to and screwing onto the outer thread on the barrel; and
an inclined annular surface defined inside the sheath between the disk and the sidewall, pressing the end tabs of the barrel inward; and
a plunger mounted slidably through the open back end of the barrel and having
a front end;
a back end;
an outer surface; and
a sealed piston made of resilient material, mounted securely around the front end of the plunger and mounted slidably in the medication chamber in the barrel.

2. The safe syringe as claimed in claim 1, wherein the tapered hub further has multiple annular nonskid ribs formed on the outer surface of the tapered hub at intervals.

3. The safe syringe as claimed in claim 1, wherein the plunger further has an annular snapping groove defined in the outer surface close to the front end of the plunger.

4. The safe syringe as claimed in claim 2, wherein the plunger further has an annular snapping groove defined in the outer surface close to the front end of the plunger.

5. The safe syringe as claimed in claim 4, wherein the barrel has three longitudinal slots and three end tabs.

6. The safe syringe as claimed in claim 5, wherein the tapered hub has four longitudinal grooves.
